(19) United States
(12) Patent Application Publication
(10) Pub. No.: US 2001/0018524 A1
(43) Pub. Date: Aug. 30, 2001

(54) Novel compounds for the management of aging-related and diabetic vascular complications, process for their preparation and therapeutic uses thereof

(76) Inventor: Alangudi Sankaranarayanan, Ahmedabad (IN)

Correspondence Address: SUGHRUE, MION, ZINN, MACPEAK & SEAS, PLLC, 2100 Pennsylvania Avenue, N.W., Washington, DC, 20037-3202, US

(21) Appl. No.: 09/801,778

(22) Filed: Mar. 9, 2001

Related U.S. Application Data
(63) Continuation-in-part of application No. 09/598,410, filed on Jun. 21, 2000.
Application No. 09/598,410 is a continuation-in-part of application No. PCT/IB99/01683, filed on Oct. 15, 1999.

(30) Foreign Application Priority Data
Oct. 6, 1999 (IN) 828/CAL /99

(51) Int. Cl. C07D 213/56

(52) U.S. Cl. 546/316

(57) ABSTRACT

Novel compounds of the pyridinium series useful for the management of diabetes and aging-related vascular complications, including kidney disease, nerve damage, atherosclerosis, retinopathy, dermatological disorders and discoloration of teeth, by breaking preformed AGE, of the general formula I, or pharmaceutically acceptable salts thereof,

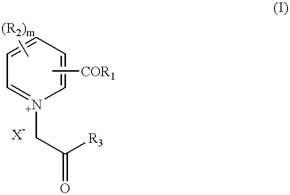

wherein, R₁, R₂, R₃, X and m are as defined in the specification. Also disclosed is a method for preparation of the compounds of general formula (I) and pharmaceutical composition containing one or more compounds as defined above as active ingredients. Also disclosed is a method of treatment of a diabetic patient by administering the compounds as defined above, either singly or in combination with drugs for antidiabetic therapy.

This is a continuation-in-part application of application Ser. No. 09/598,410 filed Jun. 21,2000, which is a continuation-in-part application of International application PCT/1B99/01683 filed on Oct., 15, 1999, the disclosures of which are incorporated herein by reference.

### BACKGROUND OF THE INVENTION

1. Field of the Invention

The present invention relates to a new class of compounds of pyridinium series and to their use in treatment of diabetes and related illnesses. More particularly the invention relates to compounds of this series, methods for their preparation, pharmaceutical composition containing these compounds and their use in the treatment of complications of diabetes mellitus. The compounds of this series exhibit AGE breaking activity, which is essential for the treatment of diabetic and aging-related complications including kidney disease, nerve damage, atherosclerosis, retinopathy and dermatological conditions. The invention also extends to the method of reversing the discoloration of teeth resulting from nonenzymatic browning in the oral cavity which comprises administration of an amount effective to reverse pre-formed advanced glycosylation crosslinks.

2. Description of the Related Art.

Maillard in 1912 found that reducing sugars, such as glucose and ribose react with proteins to form brown pigments. Further studies have shown that this is an irreversible non-enzymatic reaction, which occurs in several natural systems including stored foodstuff. Maillard reaction occurs in two stages, early and advanced. Initially, proteins react with glucose to form stable Amadori products, which subsequently cross-links to form advanced glycation end products (AGE). In most cases, the formation of AGE also accompanies browning of the proteins and increase in the fluorescence.

In diabetes, where blood glucose level is significantly higher than normal, the reaction of glucose with several proteins such as haemoglobin, lens crystallin and collagen, gives rise to the formation of AGE, which in turn, is responsible for the complications associated with diabetes, such as nephropathy, microangiopathy, endothelial dysfunction and other organ dysfunctions. In addition, the activity of several growth factors, such as basic fibroblast growth factor, is also impaired. AGE products, unlike normal proteins in tissue, have a slower rate of turnover and replenishment. It has been reported that AGE products may in fact elicit a complex immunological reaction involving RAGE (Receptor for Advanced Glycation End Products) receptors and activation of several incompletely defined immunological processes. It has been documented that diabetes with evidence of microangiopathy and macroangiopathy also show evidence of oxidative stress, the mechanism of which has not been elucidated.

In vitro AGE formation can be studied in the laboratory by incubating reducing sugars, such as ribose or glucose with bovine serum albumin. AGE formation can be detected by increase in the fluorescence or increased cross reactivity with anti-AGE antibodies. The increase in fluorescence seems to precede formation of AGE specific antigenic epitopes. This increase in fluorescence is used to monitor the increased AGE formation in vitro (Brownlee M et al, Science 1986; 232:1629-1632). In addition to the increase in the fluorescence, one of the most important features of in vitro AGE formation is the formation of antigenic epitopes that are specific to AGE and not to the native proteins. Therefore, it is possible to raise antibodies against advanced glycation end products of one protein and use them to detect AGE formation in other proteins. This has served as an important analytical tool in AGE research.

Due to the clinical significance of AGE formation, many approaches are being used to diagnose, prevent, or revert AGE formation in the body. The formation of AGE could be inhibited by reacting with an early glycosylation product that results from the original reaction between the target protein and glucose. The inhibition was believed to take place as the reaction between the inhibitor and the early glycosylation product appeared to interrupt the subsequent reaction of the glycosylated protein with additional protein material to form the cross linked late stage product. Compounds like aminoguanidine act to inhibit AGE formation by such mechanism.

The formation of AGE on long-lived proteins is also associated with cross-linking of these proteins. The AGE derived protein cross-links have been shown to be cleaved by compounds like N- phenacyl thiazolium bromide (PTB), which reacts with and cleaves covalent, AGE derived protein cross links (Vasan et al. Nature 1996; 382:275-278; U.S. Pat. No. 5,853,703, Date of Patent: Dec. 29, 1998). The mechanism of reducing the AGE content in tissues is expected to take place relatively rapidly, in contrast to aminoguanidine, which acts slowly by its very nature of mechanism of action. This current specification is related to compounds of pyridinium class, which break pre-formed AGE, like PTB, and in some cases even more effectively by than PTB.

### SUMMARY OF THE INVENTION

The main objective of the present invention is to provide a new class of compounds of the pyridinium series which are useful for the management of diabetes and aging related vascular complications and particularly in the treatment of complications of diabetes mellitus and other aging related conditions including kidney disease, nerve damage, atherosclerosis, retinopathy and dermatological conditions. The invention also extends the method to reverse the discoloration of teeth resulting from nonenzymatic browning in the oral cavity which comprises administration of an amount effective to reverse the pre-formed advanced glycosylation crosslinks, etc.

Another object of the present invention is to provide compounds of the pyridinium series, which exhibit AGE breaking activities.

Yet another object of the present invention is to provide a method of preparation of compounds of the pyridinium series which exhibit AGE breaking activities.

Still another object of the invention is to provide pharmaceutical compositions with a new class of compounds of the pyridinium series according to the invention and their pharmaceutically acceptable salts in combination with suitable carriers, solvents, excepients, diluents and other media normally employed in preparing such compositions.

Still another object of the invention is to provide a method of treatment of a diabetic patient by administration of the compounds of the invention, either singly or in combination with drugs for anti-diabetic therapy, or pharmaceutically acceptable salts thereof in required dosage in admixture with pharmaceutically acceptable diluent, solvent, excepients, carriers or other media as may be appropriate for the purpose.

### DETAILED DESCRIPTION OF THE INVENTION

The present invention provides for a new class of AGE breakers, of general formula I,

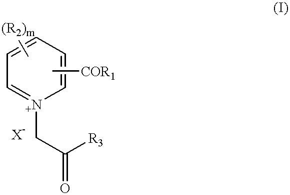

wherein

R₁ is -R₄-R₅ or -N(R₇) N(R₇) R₉;

R₄is selected from the group consisting of -N(R₇)R₆O-,-N (R7)R₆N(R₇)-, OR₆O, and -OR₆N(R₇)-, where R₆ is alkyl;

R₅ is selected from the group consisting of alkyl, aryl including heteroaryl, -COR₇, SO₂R₇, -C(S) NHR₇, -C(NH)NHR₇, -COR₁₀, —C(O)NHR₇ and

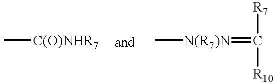

where R₇ is selected from the group consisting of H, alkyl and aryl including heteroaryl provided R₇ may be the same or different for R₁ and R₃ in the same compound;

R₂ is selected from the group consisting of F, Cl, Br, I, OR₇, NO₂, alkyl, aryl including heteroaryl, formyl, acyl, C(O)NR₇R₁₀, C(O)OR₇, NR₇R₁₀, N=C(R₇)(R₁₀), SR₇, SO₂NH₂, SO₂ alkyl and SO2aryl, and m is 0, 1 or 2;

R₃ is selected from the group consisting of R₇, OR₇, N(R₇)(R₁₀), N=C(R₇)(R₁₀), N(R₇) N(R₇)(R₁₀), N(R₇) N=C(R₇)(R₁₀) and CH(R₇)C(O)R₈ where R₈ is selected from the group consisting of R₇, OR₇ and NR₇R₁₀;

R₉ is selected from the group consisting of hydrogen, alkyl, aryl including heteroaryl, C(O)R₁₀, -SO₂R₁₀, -C(S)NHR₁₀, -C(NH) NH (R₁₀) and -C(O) NHR₁₀, R₁₀ is selected for the group consisting of H, alkyl or aryl including heteroaryl and in each case may be the same or different from substituent R₇, provided R₁₀ may be the same or different for R₁ and R₃ in the same compound;

X is selected from group consisting of a halide ion, acetate ion, perchlorate ion, sulfonate ion, oxalate ion, citrate ion, tosylate ion, maleate ion, mesylate ion, carbonate ion, sulfite ion, phosphoric hydrogen ion, phosphonate ion, phosphate ion, BF₄³¹ and PF₆³¹; with proviso that,

(i) when two alkyl groups are present on the same carbon or nitrogen, they may be linked together to form a cyclic structure and

(ii) the nitrogen of heteroaryl ring of R₁₀, when present, may be quaternized.

As used herein, “alkyl” refers to an optionally substituted hydrocarbon group joined by single carbon-carbon bonds and having 1 to 8 carbon atoms joined together. The alkyl hydrocarbon group may be linear, branched or cyclic, saturated or unsaturated. The substituents are selected from F, Cl, Br, I, N, S, O and aryl. Preferably, no more than three substituents are present.

As used herein “aryl” refers to an optionally substituted aromatic group with atleast one ring having a conjugated pi- electron system, containing upto two conjugated or fused ring systems. Aryl includes carbocyclic aryl, heterocyclic aryl and biaryl groups, all of which may be optionally substituted. The substituents are selected from F, Cl, Br, I, N, S, O and straight chain or branched C₁-C₆ hydrocarbon.

The novel compounds of the invention of general formula I having m as 0 and- COR₁ at position 3 are listed in Table 1A and the novel compounds of the invention of general formula I having m as 0 and- COR₁ at position 4 are listed in Table 1B. The following compounds suggested are by way of example alone of the representative compounds of the general formula I as defined above and in no way restrict the invention:

N,N′-bis[3-carbonyl-1-(2-phenyl-2-oxoethyl)-pyridinium] hydrazine dibromide (compound 1);

N,N′-bis[3-carbonyl-l-(2-ethoxy -2-oxoethylpyridinium]hydrazine dibromide (compound 2);

N,N′-bis[3-carbonyl-1 -(2-(2′, 4′-dichloropheny-2-oxoethyl) pyridinium]hydrazine dibromide (compound 3);

1-(2-ethoxy-2-oxoethyl) -3-(2-(2-pyridyl) hydrazinocarbonyl) pyridinium bromide (compound 4);

1-(2-thien-2′-yl-2-oxoethyl) -3-(methanesulfonylhydrazinocarbonyl) pyridinium bromide (compound 5);

N,N′-bis[3-carbonyl-1-(2-thien -2′-yl-2-oxoethyl)pyridinium] hydrazine dibromide (compound 6);

1-(2-ethoxy-2-oxoethyl) -3-(2-(benzoyloxy) ethylaminocarbonyl) pyridinium bromide (compound 7);

1-(2-(2′, 4′-dichlorophenyl) -2-oxoethyl) -3-(2-(benzoyloxy) ethylamino-carbonyl) pyridinium bromide (compound 8);

1-(2-thien-2′-yl-2-oxoethyl) -3-(2-(2-pyridyl) hydrazinocarbonyl) pyridinium bromide (compound 9);

1-(2-phenyl-2-oxoethyl) -3-(2-(2-pyridyl)hydrazinocarbonyl) pyridinium bromide (compound 10);

1-(2-phenyl-2-oxoethyl)-3-(hydrazinocarbonyl)pyridinium bromide (compound 11);

1-(2-phenyl-2-oxoethyl)-3-(methanesulfonyl hydrazinocarbonyl) pyridinium bromide (compound 12);

1-(2-ethoxy-2-oxoethyl)-3-(methanesulfonyl hydrazinocarbonyl) pyridmium bromide (compound 13);

1-(2-phenyl-2-oxoethyl) -3-(phenylsulfonylhydrazino carbonyl) pyridinium bromide (compound 14);

1-(2-phenyl-2-oxoethyl) -2-chloro-3-(phenylsulfonylhydrazino carbonyl) pyridinium bromide (compound 15);

1-(2-phenyl -2-oxoethyl) -3-(2-(acetoxy)ethyloxy carbonyl) pyridinium bromide (compound 16);

1-(2-ethoxy -2-oxoethyl) -3-(2-(benzoyloxy) ethyloxy carbonyl) pyridinium bromide (compound 17);

1 -(2-thien -2′-yl-2-oxoethyl)-4-(2-(benzoyloxy)ethylaminocarbonyl) pyridinium bromide (compound 18);

1-(2-ethoxy -2-oxoethyl) -4-(phenylsulfonyl hydrazino carbonyl) pyridinium bromide (compound 19);

1 -(2-phenylamino-2-oxoethyl)-4-(phenylsulfonyl hydrazino carbonyl) pyridinium chloride (compound 20);

1-(2-ethoxy -2-oxoethyl) -3-(phenylsulfonyl hydrazino carbonyl) pyridmium bromide (compound 21);

1-(2-(21,4′-dichlorophenyl )-2-oxoethyl)-3-(2 (methoxy)ethyloxycarbonyl) pyridmium bromide (Compound 22);

1 -(2-phenylamino-2-oxoethyl)-3-((benzoyloxy)ethylaminocarbonyl) pyridinium chloride (compound 23);

1-(2-thien-2′-yl- 2-oxoethyl)-3-(phenylaminocarbonyl hydrazinocarbonyl)pyridinium bromide (compound 24);

1 -(2-phenyl-2-oxoethyl)-3-(2-(acetoxy)ethylaminocarbonyl) pyridinium bromide (compound 25);

1-(2-phenylamino-2-oxoethyl)-3-(phenyl sulfonyl hydrazino carbonyl) pyridinium chloride (compound 26);

1-(2-phenylamino-2-oxoethyl)-3-((4-methylphenyl) sulfonyl hydrazino carbonyl)pyridinium chloride (compound 27);

1 -(2-phenyl-2-oxoethyl)-3-(2-(benzoyloxy)ethyloxy carbonyl) pyridinium bromide (compound 28);

1-(2-thien-2′-yl-oxoethyl)-3-(phenylcarbonyl hydrazino carbonyl) pyridinium bromide (compound 29);

1-(2-ethoxy-2-oxoethyl)-3-((phenylmethyl)sulfonyl hydrazino carbonyl)pyridinium bromide (compound 30);

1-(2-phenyl-2-oxoethyl)-3-((phenylmethyl)sulfonyl hydrazino carbonyl) pyridinium bromide (compound 31);

N,N′-bis [3-carbonyl-1-(2-furan-2′-yl-2-oxoethyl) pyridinium] hydrazine dibromide. (Compound No: 32);

N,N′-bis [3-carbonyl-1-(2-thien-2′-yl-2-oxoethyl) pyridinium] hydrazine dichloride.(Compound No: 33);

1-(2-thien-2′-yl-2-oxoethyl)-3-((2-(1-oxo-3-cyclohexyl)-propyl)-hydrazino carbonyl)-pyridinium bromide.( Compound No: 34);

1-(2-phenylamino-2-oxo ethyl)-3-({2-(1-oxo-3-cyclohexyl)-propyl} -hydrazino-carbonyl 3-pyridinium bromide.(Compound No: 35);

1-(2-thien-2′-yl-2-oxoethyl)-3-[2-(benzoyloxy)ethylamino carbonyl]-pyridinium bromide (Compound No: 36);

1-(4-ethoxy-2, 4-dioxobuty1)-3-(2-(benzoyloxy)ethylamino carbonyl)-pyridinium chloride. (Compound No: 37);

1-(2′, 4′-dichloro-phenyl-2-oxoethyl)-3-(2-methoxyethyl aminocarbonyl)-pyridinium bromide. (Compound No: 38);

N,,N-bis-[3-carbonyl-1-(2-cyclopropylamino-2-oxoethyl) pyridinium] hydrazine dichloride. (Compound No: 39);

1-(2-cyclopropylamino-2-oxoethyl)-3-(2-methoxyethylaminocarbonyl)-pyridinium chloride. (Compound No: 40);

N-M-bis [3-carbonyl-l-(2-isopropylamino-2-oxoethyl) pyridinium] hydrazine dichloride. (Compound No: 41);

1-(2-thien-2′yl-2-oxoethyl)3-(2-(2-chloro-3-pyridoylhydrazinocarbonyl)-pyridinium chloride. (Compound No: 42);

1-(2-isopropylamino-2-oxoethyl)-3-(2-methylsulfonylhydrazinocarbonyl)-pyridinium chloride. (Compound No: 43);

1-(2-(1-pyrrolidinyl)-2-oxoethyl)-3-(methanesulfonyl hydrazino carbonyl) pyridinium chloride.(Compound No: 44);-(2-thien-2′-yl-2-oxoethyl)-3-(methanesulfonyl hydrazino carbonyl) pyridinium chloride. (Compound No: 45);

N,N′-bis[3-carbonyl- 1-(2-hydroxy-2-oxoethyl)pyridinium]hydrazine dichloride (Compound No: 46);

1-(2-thien-2′-yl-2-oxoethyl)-3-((2-methoxy ethyl) amino carbonyl)-5-bromo pyridinium chloride (Compound No: 47);-thien-2′-yl-2-oxoethyl)-3-[1-oxo-1-(2-methoxy carbonyl)pyridyl] hydrazino pyridinium chloride. (Compound No: 48);

TABLE 1A
Representative Pyridinium derivatives
(having m as 0 and —COR₁ at position 3)

| Compound | R₁ | —R₂ | —R₃ | —X |
|---|---|---|---|---|
| 1 | Structure (a) | — | phenyl | Br |
| 2 | Structure (b) | — | OEt | Br |
| 3 | Structure (c) | — | 2,4-dichlorophenyl | Br |
| 4 | NHNH-(2-pyridyl) | — | OEt | Br |
| 5 | NHNHSO₂CH₃ | — | 2-thienyl | Br |
| 6 | Structure (d) | — | 2-thienyl | Br |
| 7 | NHCH₂CH₂OCOPh | — | OEt | Br |
| 8 | NHCH₂CH₂OCOPh | — | 2,4-dichlorophenyl | Br |
| 9 | NHNH-(2-pyridyl) | — | 2-thienyl | Br |
| 10 | NHNH-(2-pyridyl) | — | phenyl | Br |
| 11 | NHNH₂ | — | phenyl | Br |
| 12 | NHNHSO₂CH₃ | — | phenyl | Br |
| 13 | NHNHSO₂CH₃ | — | OEt | Br |
| 14 | NHNH—SO₂phenyl | — | phenyl | Br |
| 15 | NHNH—SO₂phenyl | 2-Cl | phenyl | Br |
| 16 | OCH₂CH₂OCOCH₃ | — | phenyl | Br |
| 17 | OCH₂CH₂OCOPh | — | OEt | Br |
| 21 | —NHNH—SO₂Ph | — | OEt | Br |
| 22 | —OCH₂CH₂OCH₃ | — | 2,4-dichlorophenyl | Br |
| 23 | —NHCH₂CH₂OCOPh | — | NH phenyl | CI |
| 24 | —NHNHCONHPh | — | 2-thienyl | Br |
| 25 | NHCH₂CH₂OCOCH₃ | — | phenyl | Br |
| 26 | NHNHSO₂Ph | — | NH phenyl | CI |
| 27 | NHNHSO₂Ph(4-CH₃) | — | NH phenyl | CI |
| 28 | OCH₂CH₂OCOPh | — | phenyl | Br |
| 29 | —NHNHCOPh | — | 2-thienyl | Br |
| 30 | NHNHSO₂CH₂Ph | — | OEt | Br |
| 31 | NHNHSO₂CH₂Ph | — | phenyl | Br |
| 32 | Structure (e) | H | 2-furyl | Br |
| 33 | Structure-(f) | H | 2-thienyl | Cl |
| 34 | NHNHCOCH₂CH₂- | H | 2-thienyl | Br |
| | cyclohexyl | | | |
| 35 | NHNHCOCH₂CH₂- | H | NH-phenyl | Cl |
| | cyclohexyl | | | |
| 36 | NHCH₂CH₂OCO- | H | 2-thienyl | Br |
| | phenyl | | | |
| 37 | NHCH₂CH₂OCO- | H | CH₂CO₂-ethyl | Cl |
| | phenyl | | | |
| 38 | —NHCH₂CH₂OCH₃ | H | -2,4-dichlorophenyl | Br |
| 39 | Structure-(g) | H | NH-cyclopropyl | Cl |
| 40 | —NHCH₂CH₂OCH₃ | H | NH-cyclopropyl | Cl |
| 41 | Structure-(h) | H | NH-isopropyl | Cl |
| 42 | Structure-(i) | H | 2-thienyl | Cl |
| 43 | NHNHSO₂CH₃ | H | NH-isopropyl | Cl |
| 44 | NHNHSO₂CH₃ | H | 1-pyrrolidinyl | Cl |
| 45 | NHNHSO₂CH₃ | H | 2-thienyl | Cl |
| 46 | Structure-(j) | H | —OH | Cl |
| 47 | NHCH₂CH₂OCH₃ | H | 2-thienyl | Cl |
| 48 | Structure-(k) | H | 2-thienyl | Cl |

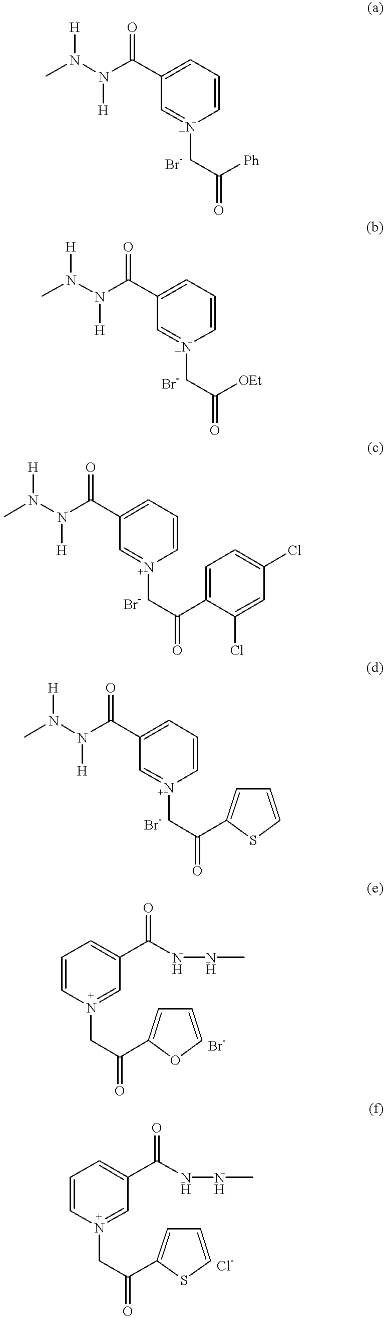 TABLE 1B
Representative Pyridinium derivatives
(having m as 0 and —COR₁ at position 4)

| Compound | —R₁ | —R₂ | —R₃ | —X |
|---|---|---|---|---|
| 18 | NHCH₂CH₂OCOPh | — | 2-thienyl | Br |
| 19 | NHNHSO₂Ph | — | OEt | Br |
| 20 | NHNHSO₂Ph | — | NH phenyl | CI |

According to the embodiment of the present invention, the present compounds are used for the treatment of diabetic complications, and aging related complications including kidney disease, nerve damage, atherosclerosis, retinopathy, dermatological conditions and colouration of teeth occurring due to the higher levels of preformed AGE. The increased levels of preformed AGE can be brought under control by breaking the AGE products using compounds mentioned in the invention.

The invention also provides a process for the preparation of novel compounds of the pyridinium series.

The said process for the preparation of compound 1, comprises, adding a solution of phenacyl bromide in isopropanol to N,N′-bis-(nicotinyl) hydrazine dissolved in methanol, refluxing for six hours, cooling, filtering the precipitated solid, washing the solid with hot ethyl acetate and finally purifying the solid with 20 ml of methanol: ethyl acetate (3:1) to yield the desired compound.

Similarly, the other novel compounds of general formula I, are prepared from properly substituted pyridine derivatives followed by quarternization with appropriate reagent by refluxing in alcoholic solvents like, methanol, ethanol, propanol, etc and high boiling solvents like toluene or xylene etc, for 6-48 hrs. to give the desired compounds.

The examples of substituted pyridine derivatives which can be used for preparation of specific compounds of the invention are given below:

1. N,N′-bis(nicotinyl)hydrazine

2. 3-[(2-pyridyl)hydrazinocarbonyl]pyridine

3. 3-[2-methanesulfonyl)hydrazinocarbonyl]pyridine

4. 3-[(2-benzoyloxy)ethylaminocarbonyl]pyridine

5. 3-[(2-phyenylsulfonyl)hydrazinocarbonyl]pyridine

6. 3-[(2-acetoxy)ethyloxycarbonyl]pyridine

7. 3-[(2-benzoyloxy)ethyloxycarbonyl]pyridine

8. 3-[(2-methoxy)ethyloxycarbonyl]pyridine

9. 3-[(2-phyenylamninocarbonyl)hydrazinocarbonyl]pyridine

10. 3-[(2-acetoxy)ethylaminocarbonyl]pyridine

11. 3-[(2-(4-methylphenyl sulfonylhydrazinocarbonyl))]pyridine

12. 3-[(2-benzoyl)- hydrazino carbonyl]pyridine

13. 3-[(2-phenylmethane sulfonyl) hydrazino carbonyl]pyridine

14. 3-[(2-(3-cyclohexylpropanoyl) hydrazino carbonyl]pyridine

15. 3-[(2-methoxy)ethylaminocarbonyl]pyridine

16. 3-[1-oxo-1-(2-methoxycarbonyl)pyridyl]hydrazino pyridine

The examples of quaternizing agents which may be used in the reaction are given below:

1. 2-bromoacetyl thiophene

2. 2-chloroacetyl thiopene

3. phenacylbromide

4. phenacylchloride

5. 2,4-dichloropheanacylbromide

6. N- phenyl chloroacetamide

7. N- cyclopropyl chloroacetamide

8. ethylbromoacetate

9. bromo acetylfuran

10. N- isopropylchloroacetamide

11. N- chloroacetyl-2-pyrrolidinone

12. chloroacetic acid

### In-vitro screening for AGE-breaking Activity

The in vitro AGE formation, studied in the laboratory, by incubating reducing sugar glucose, with protein bovine serum albumin, resulted in browning of solution and increase in the fluorescence. Fluorescence was used as the criteria to monitor the increased AGE formation.

### EXAMPLE 1

### AGE breaker activity has been confirmed by the screening procedure as mentioned below:

Materials:

Bovine serum albumin (fraction V) (BSA)

Glucose, analytical grade

Phosphate buffered saline (PBS)

Equipment:

Microplate ELISA Reader- Spectramax Plus (Molecular Devices, USA)

Microplate washer, (Bio -Tec Instruments, USA)

pH meter

Methods of experiment: Elisa (Enzyme Linked Immunosorbent Assay)

160 mg/ml of protein, bovine serum albumin, BSA and 1.6M glucose sugar were dissolved in phosphate buffered saline, PBS. Sodium azide was added at 0.02% concentration as a preservative. The solution was filtered asceptically through a 0.22 μM filter and kept for aging at 37° C. for 16 weeks. After 16 weeks the solution was dialyzed against PBS, aliquoted and stored at −20° C.

To determine the AGE breaking activity, 10μg/ml of the 16 weeks AGE-BSA was incubated with different concentrations of the test compounds at 37° C. for 24 hours and AGE breaking activity of the test compounds by ELISA was determined.

ELISA was performed as follows:

1. Different concentrations of 16 weeks AGE-BSA were coated on a microtitre plate as standard. Each concentration is coated in triplicates.

2. The test samples were coated on microtitre plate at a concentration of 5 ng. to 20 ng per well in triplicates.

3. The plate was incubated at 37° C. for one hour.

4. After incubation the plate was washed with PBST (PBS with 0.05% Tween 20).

5. Blocking with 5% skimmed milk in PBS at 37° C. for one hour was done.

6. The plate was washed with PBST.

7. Primary antibody against AGE-BSA was added and the plate is incubated at 37° C. for one hour.

8. The plate was washed with PBST

9. Secondary antibody anti rabbit HRPO (Horse-Radish Per Oxidase) conjugate was added and the plate is incubated at 37° C. for one hour.

10. The plate was washed with PBST.

11. Colour development with OPD (orthophenylenediamine dihydrochloride) and hydrogen peroxide was done.

12.OD (optical density) at (450 nm reading-620 nm reading) was measured after incubation at 37° C. for 15 minutes with Microplate ELISA Reader.

The breaker activity of the compounds were determined by the following formula:

$$\% Breaker activity = \frac{{OD}_{450 - 620} Control - {OD}_{450 - 620} Test}{{OD}_{450 - 620} Control}$$

OD₄₅₀₋₆₂₀Control=Absorbance of 20 ng AGE-BSA after incubation at 37° C. for 24 hours without test compound OD₄₅₀₋₆₂₀Test=Absorbance of 20 ng AGE-BSA after incubation at 37° C. for 24 hours with required concentration of test compound Using specific examples, the % AGE breaking activity was calculated and recorded in Table 2.

TABLE 2

| Sample | Concentration | | % Breakage |
|---|---|---|---|
| PTB | 10 | mM | 27 |
| | 20 | mM | 47 |
| Compound 1 | 5 | mM | 13 |
| Compound 4 | 10 | mM | 30 |
| Compound 5 | 10 | mM | 16 |
| | 50 | mM | 68 |
| Compound 6 | 5 | mM | 53 |

| | | |
|---|---|---|
| Compound 7 | 20 mM | 36 |
| Compound 16 | 10 mM | 16 |
| Compound 17 | 10 mM | 19 |
| Compound 22 | 10 mM | 13 |
| | 25 mM | 41 |
| Compound 23 | 10 mM | 37 |
| | 25 mM | 90 |
| Compound 32 | 10 mM | 14 |
| Compound 33 | 5 mM | 20 |
| Compound 38 | 5 mM | 17.66 |
| Compound 39 | 5 mM | 22.8 |
| Compound 40 | 10 mM | 12.38 |
| Compound 42 | 10 mM | 12.51 |
| Compound 43 | 10 mM | 10.85 |
| Compound 45 | 10 mM | 17.53 |
| Compound 47 | 10 mM | 32.38 |

Hence compound 6 has significant AGE breaking activity i.e. a comparatively much superior potency vis-a-vis PTB.

### In-vivo screening for AGE-breaking Activity:

The test compounds were studied for their beneficial effects on diabetic neuropathy and nephropathy in a rat model of diabetes. The rats were divided into three groups. The first group consisted of age matched untreated non-diabetic animals. The second group consisted of diabetic controls and the third group was the diabetic group treated with the test compound. Each treatment group had its own corresponding control and diabetic groups. The second and third groups were treated with Streptozotocin (STZ) at 60 mg/kg for the induction of diabetes. After completion of 12 weeks of diabetes the rats were treated with the test compound daily (doses shown in table) for a period of 8 weeks. At the end of the treatment the creatinine clearances and nerve conduction velocities (NCV) of the animals were estimated.

Creatinine clearances of the rats were estimated as follows

Creatinine clearance

=Concentration of creatinine in the urine×ml urine passed/minute

Concentration of creatinine in the blood.

The creatinine clearance in untreated diabetic group was compared with the treated group and the percentage improvements are shown in the Table 3.

The nerve conduction velocity was measured using a modified method of Biro et al 1998. Briefly under ether anesthesia the sciatic and tibial nerves were electrically stimulated at the sciatic notch or ankle, respectively. Electromyograms (EMG's) recorded from the plantar muscles consisted of two components: (1) the short latency direct motor response (M) and the monosynaptically elicited long-latency sensory response (H, Hoffmann reflex). Latency and the duration of the M responses were measured and the motor nerve conduction velocity (MNCV) was calculated as follows:

$$MNCV = \frac{Distance between the sciatic and tibial stimulation points}{Differences of the latency for {M}_{sciatic} and {M}_{tibial}} .$$

The percentage improvement in the nerve conduction velocities in the group treated with the test compounds was calculated as follows:

$$\begin{matrix}\% Improvement \\ in the NCV ' s\end{matrix} = \frac{\begin{matrix}NCV of the treated group - \\ NCV of the diabetic group\end{matrix}}{\begin{matrix}NCV of the control group - \\ NCV of the diabetic group\end{matrix}} .$$

TABLE 3
Effect of compound Nos 33 and 39 on the creati-
nine clearance and nerve conduction velocities:

| | Compound No. 33 | Compound No. 39 |
|---|---|---|
| Parameters | (7.5 mg/kg, b.i.d.) | (6.0 mg/kg, b.i.d.) |
| % Increase in creatinine | 103.0 | 5.0 |
| clearance | | |
| % Increase in the NCV | 60.0 | 58.4 |

The results show that compounds of this class have beneficial effects on creatinine clearance and nerve conduction velocities.

### Discussion of the test results:

All the test compounds mentioned in the current application have shown an invitro AGE-breaker effect. Under conditions of chronic hyperglycemia in rats there is a spontaneous non-enzymatic reaction between glucose, lipids and proteins that leads to the formation of advanced glycosylation end products. In this animal model decreased creatinine clearance and decreased nerve conduction velocity have been demonstrated. These changes are related to damage to renal and neuronal tissues. During chronic NIDDM patients, there is a decrease in the creatinine clearances as a manifestation of the diabetes induced renal damage. One of the major factors contributing to renal damage is the glycation of the long-lived proteins in the kidney. It is well recognized that there is a decrease in the nerve conduction velocities in chronic diabetic subjects, which is a manifestation of neuropathy. Breaking of cross-linked proteins in the neuronal tissues and associated vasculature could lead to an improvement in the neuronal function.

The compounds of the present invention have shown a functional improvement both in terms of the improvement in the creatinine clearance and an improvement in the nerve conduction velocities. The evidences stated above clearly demonstrate that these compounds could play a major role in the prevention and treatment of various diabetic and aging related complications like nephropathy and neuropathy.

The following examples give method of preparation of the specific novel compounds of the invention as given in Table 1. The following compounds suggested are by way of example alone and in no way restrict the invention.

### EXAMPLE 2

### Preparation of N,N′-bis [3-carbonyl-1-(2-phenyl-2-oxoethyl) pyridinium]hydrazine dibromide (compound 1):

To a boiling solution of N,N′-bis-(nicotinyl)hydrazine (1.21 g., 0.005 mol.) in methanol (20 ml.), a solution of phenacyl bromide (1.99 g., 0.01 mol.) in isopropanol (10 ml.) was added and the reaction mixture was refluxed for 6 hrs. The reaction mixture was concentrated under vacuum (˜10 ml.) and filtered. The obtained residue was washed with hot ethylacetate and then the isolated solid was powdered. It was recrystallised from a mixture of methanol and ethylacetate (3:1, 20 ml) to afford a pale yellow solid.

Yield: 60%

m.p.:260-262° C. (decomp.)

IR (KBr, cm⁻¹): 1696 and 1680

¹HNMR (DMSOd₆, 400MHz)δ:11.65(2H,s), 9.56(2H,s), 9.21-9.16(4H,m), 8.49-8.45(2H,m), 8.08-8.05(4H,d), 7.81-7.77(2H,m), 7.68-7.64(4H,m), 6.58(4H,s)

Mass (m/z):479, 480

According to the above mentioned procedure the following compounds are synthesized by reacting the corresponding pyridine derivatives with appropriate reagents by refluxing in methanol, ethanol, propanol, toluene or xylene for 6-48 hrs. to get the desired compounds:

### EXAMPLE 3

### N,N′-Bis[3-carbonyl-1-(2-ethoxy -2-oxoethyl) pyridinium] hydrazine dibromide (compound 2):

Yield: 47%

m.p.: 180-182° C. (decomp.)

IR (KBr, cm⁻¹): 1744, 1664

¹HNMR (DMSOd₆, 400MHz)δ:11.65(2H,s), 9.62(2H,s), 9.28-9.26(2H,d), 9.17-9.15(2H,d), 8.47-8.44(2H,m), 5.77(4H,s), 4.26(4H,q), 1.27(6H,t)

Mass (m/z):415, 416

### EXAMPLE 4

### N,N′-Bis[3-carbonyl-1-(2-(2,4-dichlorophenyl) -2-oxoethyl) pyridinium] hydrazine dibromide (compound 3):

Yield:24%

m.p.:225-227° C. (decomp.)

IR (KBr, cm⁻¹):1702, 1666

¹HNMR (DMSOd₆, 400 MHz)δ:11.69(2H,s), 9.58(2H,bs), 9.20-9.18(4H,m), 8.49-8.47(2H,m), 8.17-8.15(2H,d), 7.92(2H,bs), 7.78-7.76(2H,d), 6.50(4H,s)

Mass (m/z): 615, 617, 618, 620.

### EXAMPLE 5

### 1-(2-Ethoxy -2-oxoethyl)-3-(2-(2-pyridyl) hydrazinocarbonyl pyridinium bromide (compound 4):

Yield: 16%

m.p.:210-212° C.

IR (KBr, cm⁻¹):3140, 3005, 1732 and 1690

¹HNMR (DMSOd₆, 400MHz)δ:9.63(1H,s), 9.27(2H,d), 8.49-8.45(1H,m)8.13-8.07(2H,m), 7.32-7.30(1H,m), 7.12-7.11(1H,m), 5.77(2H,s), 4.23(2H,q), 1.25(3H,t)

Mass (m/z):301, 302

### EXAMPLE 6

### 1-(2-Thien -2′-yl-2-oxoethyl) -3-(methanesulfonyl hydrazinocarbonyl) pyridinium bromide (compound 5):

Yield: 30 %

m.p.:199-200° C.

IR (KBr, cm⁻¹):1714, 1673

¹HNMR ([DMSOd₆, 400 MHz)δ:11.38(1H,s), 9.97(1H,s) 9.51(1H,s), 9.16(1H,d), 9.06-9.04(1H,m), 8.43-8.39(1H,m), 8.25-8.21(2H,m), 7.43-7.41(1H,t), 6.45(2H,s), 3.08(3H,s).

Mass (m/z) 340, 341, 342

### EXAMPLE 7

### N,N′-Bis[3-carbonyl-1-(2-thien -2′-yl-2-oxoethyl) pyridinium] hydrazine dibromide (compound 6):

Yield: 33%

m.p.:259-261° C. (decomp.)

IR (KBr, cm⁻¹ ):3330, 1702, 1674, 1655 and 1626

¹HNMR (DMSOd₆, 400 MHz)δ:11.59(2H,s), 9.50(2H,s), 9.15-9.08(4H,m), 8.40-8.36(2H,m), 8.17-8.14(4H,m), 7.33(2H,t), 6.42(4H,s)

Mass (m/z) : 491, 492.

### EXAMPLE 8

### 1-(2-Ethoxy -2-oxoethyl) -3-(2-(benzoyloxy) ethylaminocarbonyl) pyridinium bromide (compound 7):

Yield: 85%

m.p.:132-134° C.

IR (KBr, cm⁻¹):3210, 3067, 1726, 1687, 1656

¹HNMR (DMSOd₆, 400 MHz)δ:9.46 (1H,s), 9.37(1H,t), 9.11(1H,t), 8.97(1H,d), 8.33-8.29(1H,m) 7.95-7.93(2H,m), 7.63-7.59(1H,m), 7.49-7.45(2H,m), 5.65(2H,s), 4.39(2H,t), 4.19(2H,q), 3.70-3.69(2H,m), 1.20(3H,t)

Mass(m/z):357,358,359

### EXAMPLE 9

### 1-(2-(2′, 4′-Dichlorophenyl) -2-oxoethyl) -3-(2-(benzoloxy)ethyl aminocarbonyl) pyridinium bromide (compound 8):

Yield: 75%

m.p.:102-104° C.

IR (KBr, cm⁻¹):1703, 1685, 1675

¹HNMR (DMSOd₆, 400 MHz)δ:9.41-9.37(2H,m), 9.03-8.98(2H,m)8.34-8.30(1H,m), 8.04(1H,d), 7.91-7.89(2H,m), 7.82 (1H,d),7.68-7.65(1H,m), 7.58-7.55(1H,m), 7.43(2H,t), 6.35(2H,s), 4.36(2H,t), 3.68-3.64(2H,m)

Mass (m/z):457, 458, 459, 460, 461, 462

### EXAMPLE 10

### 1-(2-Thien -2′-yl -2-oxoethyl) -3-(2-(2-pyridyl)hydrazinocarbonyl) pyridinium bromide (compound 9):

Yield: 10%

m.p.:212-214° C. (decomp)

IR (KBr, cm⁻¹):1685, 1649

¹HNMR (DMSOd₆, 400 MHz)δ:11.21(1H,bs), 9.59(1H,s), 9.19(2H,d), 8.44(1H,t), 8.27-8.24(2H,m), 8.08(1H,bs), 7.62(1H,bs), 7.44(1H,t), 6.85-6.79(2H,m), 6.50(2H,s)

Mass (m/z):339, 340, 341

### EXAMPLE 11

### 1-(2-Phenyl-2-oxoethyl)-3-(2-(2-pyridyl) hydrazinocarbonyl) pyridinium bromide (compound 10):

Yield: 4%

m.p.:190° C. (decomp)

IR (KBr, cm⁻¹):1683, 1670, 1648

¹HNMR(DMSOd₆, 400 MHz)δ:11.14(1H,bs), 9.53(1H,s), 9.18-9.13(2H,m), 8.45-8.42(1H,t), 8.08-8.06(3H,m), 7.80(1H,t), 7.67(2H,t), 7.62-7.55(1H,m), 6.83-6.76(2H,m), 6.54(2H,s)

Mass (m/z):333, 334, 335

### EXAMPLE 12

### 1-(2-Phenyl-2-oxoethyl) -3-(hydrazinocarbonyl) pyridinium bromide (compound 11).

Yield: 15%

m.p.:215 -216° C.

IR (KBr, cm⁻¹):1695, 1680

¹HNMR (DMSOd₆, 400 MHz)δ:10.25(1H,s) 9.65(1H,s), 9.35-9.32(2H,m), 8.90-8.88(1H,m)8.50-8.46(2H,d), 8.21-8.17(1H,m), 8.05-8.07(2H,m), 6.50(2H,s), 4.45(2H,s).

Mass (m/z):256, 257.

### EXAMPLE 13

### 1-(2-Phenyl-2-oxoethyl)-3-(methanesulfonyl hydrazinocarbonyl) pyridinium bromide (compound 12):

Yield: 35%

m.p.:227-228° C.

IR (KBr, cm⁻¹):1710, 1702

¹HNMR (DMSOd₆, 400 MHz)δ:11.30,(1H,s), 9.88(1H,s), 9.41(1H,s), 9.06-9.05(1H,d)8.98-8.96(1H,d), 8.34-8.31(1H,m), 7.97(2H,d), 7.72-7.69(1H,t), 7.59-7.56(2H,t), 6.44(2H,s), 2.99(3H,s)

Mass (m/z):334, 335

### EXAMPLE 14

### 1-(2-Ethoxy-2-oxoethyl)-3-(methanesulfonyl hydrazinocarbonyl) pyridinium bromide (compound 13):

Yield: 38%

m.p.:75-76° C.

IR (KBr, cm⁻¹):1739, 1697

¹HNMR(DMSOd₆, 400 MHz)δ:11.39(1H,s), 9.96(1H,s), 9.56(1H,s), 9.23(1H,d), 9.06(1Hd), 8.40(1H,t), 5.75(2H,s), 4.27-4.22(2H,q), 3.08(3H,s), 1.26(3H,t)

Mass (m/z): 301, 302, 303

### EXAMPLE 15

### 1-(2-Phenyl-2-oxoethyl)-3-(phenylsulfonylhydrazino carbonyl) pyridinium bromide (compound 14):

Yield: 28%

m.p.:187-188° C.(dec.)

IR (KBr, cm⁻¹):1700, 1633

¹HNMR(DMSOd₆, 400 MHz)δ:11.38(1H,s), 10.45(1H,s), 9.33(1H,s), 9.13-9.12(1H,d), 8.95(1H,d), 8.38(1H,t), 8.05(2H,d), 7.89(2H,d), 7.80(1H,t), 7.66(3H,t), 7.57(2H,t), 6.50(2H,s).

Mass (m/z):396, 397, 398

### EXAMPLE 16

### 1-(2-Phenyl-2-oxoethyl)2-chloro-3-(phenylsulfonylhydrazino carbonyl) pyridinium bromide (compound 15):

Yield: 23%

m.p.:247-250° C. (decomp)

IR (KBr, cm⁻¹):1685, 1679,

¹HNMR(DMSOd₆, 400 MHz)δ:11.12(1H,s), 9.49(1H,s), 9.07-9.03(1H,m), 8.44(1H,t), 8.07(2H,d), 7.80(1H,t), 7.67(2H,t), 7.18(2H,t), 6.87(2H,d), 6.77(1H,t), 6.50(2H,s).

Mass (m/z):430, 431, 432

### EXAMPLE 17

### 1-(2-Phenyl-2-oxoethyl) -3-(2-(acetoxy)ethyloxy carbonyl) pyridinium bromide (compound 16):

Yield: 40%

m.p.:152-153° C.

IR (KBr, cm⁻¹) :1737, 1691, 1635

¹HNMR(DMSOd₆, 400 MHz)δ:9.63(1H,s), 9.24(1H,d), 9.12(1H,d), 8.43(1H,t), 8.07(2H,d), 7.80(1H,t), 7.67(2H,t), 6.59(2H,s), 4.62-4.60(2H,m), 4.39-4.37(2H,m), 2.03(3H,s)

Mass (m/z):328, 329

### EXAMPLE 18

### 1-(2-Ethoxy -2-oxoethyl) -3-(2-(benzoyloxy) ethyloxycarbonyl) pyridinium bromide (compound 17):

Yield:35%

m.p.:142-143° C.

IR (KBr, cm¹):1736, 1718, 1636

¹HNMR (DMSOd₆, 400 MHz)δ:9.60(1H,s), 9.20-9.18(1H,d), 9.04-9.02(1H,d), 8.33-8.29(1H,m), 7.90-7.88(2H,d), 7.58-7.57(1H,m), 7.46-7.42(2H,m), 5.67(2H,s), 4.71-4.68(2H,m), 4.58-4.56(2H,m), 4.15(2H,q), 1.16(3H,t)

Mass (m/z):358,359,360

### EXAMPLE 19

### 1-(2-Thien -2′-yl-2-oxoethyl)-4-(2-(benzoyloxy) ethylaminocarbonyl) pyridinium bromide (compound 18):

m.p.:210-211° C.

IR (KBr, cm⁻¹):1723, 1680, 1668

¹HNMR (DMSOd₆, 400 MHz)δ:9.52(1H,t), 9.14(2H,d), 8.50(2H,d), 8.25-8.21(2H,m), 8.01-7.99(2H,d), 7.67(1H,t), 7.55-7.51 (2H,m), 7.42-7.40(1H,m), 6.42(1H,s) 4.47-4.45(2H,t), 3.77-3.73(2H,m).

Mass (m/z):395, 396

### EXAMPLE 20

### 1-(2-Ethoxy-2-oxoethyl)-4-(phenylsulfonyl hydrazino carbonyl) pyridinium bromide (Compound 19):

Yield:60%

m.p.:171-173° C.

IR (KBr, cm⁻¹):1745, 1685, 1645.

¹HNMR (DMSOd₆, 400 MHz)δ:11.41(1H,s), 10.39(111, s), 9.10(2H,d), 8.27(2H,d), 7.82-7.80(2H,d), 7.60-7.57(1H,t), 7.50-7.46(2H, t), 5.63(2H,s), 4.18-4.12(2H,q), 1.19-1.15(3H,t).

Mass (m/z):364, 365, 366

### EXAMPLE 21

### 1-(2-Phenylamino-2-oxoethyl)-4-(phenylsulfonyl hydrazino carbonyl) pyridinium chloride (Compound 20):

Yield:10%

m.p.:225-227° C.

IR (KBr, cm⁻¹):1693, 1642, 1592

¹HNMR(DMSOd₆, 400 MHz)δ:11.55(1H,s), 10.99(1H,s), 10.49(1H,s), 9.20(2H,d), 8.34(2H,d), 7.89(2H,d), 7.73-7.64(1H,t), 7.61-7.56(4H,m), 7.37-7.33(2H,t), 7.12-7.09(1H,t), 5.73(2H,s).

Mass (m/z):411, 412, 413, 414

### EXAMPLE 22

### 1-(2-Ethoxy-2-oxoethyl)-3-(phenylsulfonylhydrazino carbonyl) pyridinium bromide (Compound 21):

Yield:75%

m.p.:145-147 ° C.

IR (KBr cm⁻¹):1744, 1713, 1633

¹HNMR (DMSOd₆, 400 MHz)δ:11.27(1H,s),10.36(1H,s), 9.28(1H,s), 9.09(1H,d), 8.83(1H,d),8.27-8.24(1H,m), 7.82-7.79(2H,m), 7.58(1H,t), 7.48(2H,t), 5.59(2H,s), 4.17-4.12(2H, q), 1.16(3H,t).

Mass (m/z):364, 365, 366

### EXAMPLE 23

### 1-(2-(2′, 4′-Dichlorophenyl)-2-oxoethyl)-3-(2(methoxy)ethyloxycarbonyl) pyridinium bromide (Compound 22):

Yield:25%

m.p.:156-158° C.

IR (KBr, cm⁻¹):1731, 1706, 1640

¹HNMR (DMSO d₆, 400 MHz)δ:9.61(1H,s),9.20(1H,d),9.13(1H,d), 8.45-8.41(1H,m),8.15(1H,d),7.92(1H,d),7.78-7.76(1H,m), 6.49(2H,s), 4.56-4.54(2H,m), 3.72-3.69(2H, q), 3.31(3H,s).

Mass (m/z):368, 369, 370, 371

### EXAMPLE 24

### 1-(2-Phenylamino-2-oxoethyl)-3-(2-(benzoyloxyl) ethylaminocarbonyl) pyridinium chloride (Compound 23):

Yield:70%

m.p.:171-172° C.

IR (KBr, cm⁻¹):1720, 1692, 1668

¹HNMR:(DMSOd₆, 400 MHz)δ:11.06(1H,s), 9.67(1H,t), 9.59(1H,s), 9.20(1H,d), 9.11(1H,d), 8.36-8.32(1H,m), 8.00(2H,d), 7.66-7.61 (3H,m),7.51(2H,t),7.34(2H,t), 7.10(1H,t), 5.77(2H,s), 4.45(2H,t), 3.76-3.72(2H,q).

Mass (m/z):404, 405, 406, 407

### EXAMPLE 25

### 1-(2-Thien-2′-yl-2-oxoethyl)3-(phenylaminocarbonyl hydrazinocarbonyl) pyridinium bromide (Compound 24):

Yield:30%

m.p.:202-204° C.

IR (KBr, cm⁻¹):1718, 1673

¹HNMR:(DMSOd₆, 400 MHz)δ:11.03(1H,s), 9.55(1H,s), 9.18(11H,d), 9.10(1H,d), 9.00(1H,s),8.57(1H,s), 8.46-8.42(1H,t), 8.25-8.22(2H,m), 7.47-7.45(2H,d), 7.43-7.41(1H,t), 7.29-7.25 (2H,t), 7.0-6.96(11H,t), 6.46(2H,s).

Mass (m/z):381, 382, 383

### EXAMPLE 26

### 1-(2-Phenyl-2-oxoethyl)3-(2-(acetoxy) ethylaminocarbonyl) pyridinium bromide (Compound 25):

Yield:55%

m.p.:186-188

IR (KBr, cm⁻¹):1734, 1697, 1679

¹HNMR (DMSOd₆, 400 MHz)δ:9.47(1H,s), 9.36(1H,t), 9.13-9.05(2H,m), 8.42-8.38(1H,m), 8.06(2H,d), 7.80(1H,t), 7.67(2H,t), 6.54(2H,s), 4.18(2H,t), 3.61-3.57(2H,q), 2.02(3H,s).

Mass (m/z):327, 328, 329.

### EXAMPLE 27

### 1-(2-Phenylamino-2-oxoethyl)-3-(phenyl sulfonyl hydrazino carbonyl) pyridinium chloride (Compound 26):

Yield:38%

m.p.:232-234° C.

IR (KBr, cm⁻¹):1689, 1636, 1596

¹HNMR (DMSOd₆, 400 MHz)δ:11.30(1H,s), 10.80(1H,s), 10.37(1H,s), 9.29(1H,s), 9.09(1H,d), 8.81(1H,d), 8.25-8.21(1H,t), 7.82-7.80(2H,d), 7.59-7.46(5H,m), 7.28-7.24(2H,t), 7.04-7.00 (1H,t), 5.62(2H,s).

Mass (m/z):411, 412, 413, 414

### EXAMPLE 28

### 1-(2-Phenylamino-2-oxoethyl)-3-((4-methylphenyl)sulfonyl hydrazino carbonyl) pyridinium chloride (Compound 27):

Yield:48%

m.p.:205-206° C.

IR (KBr, cm⁻¹):1712, 1681, 1632

¹HNMR (DMSOd₆, 400 MHz)δ:11.35(1H,s), 10.86(1H,s), 10.36(1H,s), 9.38(1H,s), 9.17(1H,d), 8.90(1H,d), 8.34-8.30(1H,m), 7.78-(2H,d), 7.59(2H,d), 7.37-7.33(4H,m), 7.11(1H,t), 5.70(2H,s), 2.36(3H,s).

Mass (m/z):425, 426, 427, 428

### EXAMPLE 29

### 1-(2-Phenyl-2-oxoethyl)-3-(2-(benzoyloxy)ethyloxy carbonyl) pyridinium bromide (Compound 28):

Yield:35%

m.p.:132-134° C.

IR (KBr, cm⁻¹):1730, 1705, 1690

¹HNMR (DMSOd₆, 400 MHz)δ:9.80(1H,s), 9.36(1H,d), 9.30(1H,d), 8.58(1H,t), 8.21(2H,d), 8.12(2H,d), 7.95(1H,t), 7.85-7.80(3H,m), 7.68(2H,t), 6.71(2H,s), 4.95-4.93(2H,m), 4.82-4.80(2H,m).

Mass (m/z):390, 391, 392.

### EXAMPLE 30

### 1-(2-Thien-2′-yl-2-oxoethyl)-3-(phenylcarbonyl hydrazino carbonyl) pyridinium bromide (Compound 29):

Yield:45%

m.p.:80-81 ° C.

IR (KBr Cm⁻¹) 1700, 1663, 1631

¹HNMR (DMSOd₆, 400 MHz)δ:11.49(1H,s), 10.95(1H,s), 9.67 (1H,s), 9.34(1H,d), 9.27(1H,d), 8.52-8.48(1H,m), 8.29-8.28(2H,m), 8.00(2H,d), 7.68(1H,t), 7.59(2H,t), 7.46(1H,t), 6.63(2H,s)

Mass (m/z):366, 367, 368, 369

### EXAMPLE 31

### 1-(2-Ethoxy-2-oxoethyl)-3-((phenylmethyl)sulfonyl hydrazino carbonyl) pyridinium bromide (Compound 30):

Yield:50%

m.p.:147-148° C.

IR (KBr, cm⁻¹):1749, 1698, 1640

¹HNMR (DMSOd₆, 400 MHz)δ:11.57(1H,s), 10.21(1H,s), 9.75(1H,s), 9.38(1H,d), 9.24(1H,d), 8.59-8.56(1H,m), 7.67-7.65(2H,m), 7.58-7.52 (3H,m), 5.90(2H,s),4.68(2H,s), 4.45-4.39(2H,q),1.43(3H,t).

Mass (m/z):377, 378, 379

### EXAMPLE 32

### 1-(2-Phenyl-2-oxoethyl)-3-((phenylmethyl)sulfonyl hydrazino carbonyl)pyridinium bromide (Compound 31):

Yield:80%

m.p.:205-207° C.

IR (KBr, Cm⁴):1687, 1637

¹HNMR (DMSOd₆, 400 MHz)δ:11.59(1H,s), 10.20(1H,s), 9.71(1H,s), 9.33(1H,d), 9.27(1H,d), 8.62-8.59(1H,m), 8.25-8.23(2H,d), 7.99-7.95(1H,t), 7.86-7.82(2H,t), 7.67-7.65(2H,m), 7.57-7.52 (3H,m),6.72(2H,s), 4.69(2H,s).

Mass (m/z):410, 411, 412, 413

### EXAMPLE 33

### N,N′-Bis 13-carbonyl-1-(2-furan-2′-yl-2-oxoethyl) pyridinium] hydrazine dibromide. (Compound No:32)

Yield:23%

m.p.:267-269° C. (dec)

IR (KBr, cm⁻¹):1687, 1660

¹HNMR (DMSO d₆, 400 MHz)δ:11.65 (2H,s), 9.56(2H,s), 9.21-9.15(4H,m), 8.48-8.44(2H,t), 8.23(2H,s, 7.74-7.73(2H,d), 6.91-6.90 (2H,d)6.34(4H,s)

Mass (m/z):459,460,461

### EXAMPLE 34

### N,N′-Bis [3-carbonyl -1-(2-thien-2′-yl-2-oxoethyl) pyridinium hydrazine dichloride.(Compound No:33)

Yield:35%

m.p.:275-277° C.

IR (KBr, cm⁻¹):3374, 1665,1632, 1410

¹HNMR (DMSO d₆, 400 MHz)δ:11.88(2H,s), 9.66(2H,s), 9.29-9.24 (4H,m), 8.48-8.44(2H,m), 8.25-8.23(4H,m), 7.43-7.41(2H,m), 6.53(4H,s).

Mass (m/z):491,492,493, 494

### EXAMPLE 35

### 1-(2-Thien-2′-yl-2-oxoethyl)-3-((2-(1-oxo-3-cyclohexyl)-propyl)-hydrazino carbonyl)-pyridinium bromide( Compound No:34);

Yield:15%

m.p.:217-219° C. ( dec)

IR (KBr, cm⁻¹):3190, 1708, 1667and 1404

¹HNMR (DMSO d₆, 400 MHz)δ:11.07(1H,s), 10.22(1H,s), 9.51(1H,s), 9.16-9.15(1H,d), 9.06-9.04(1H,d), 8.42-8.40(1H,m), 8.25-8.21 (2H,m), 7.43-7.40(1H,m), 6.44(2H,s), 2.25-2.22(2H,t), 1.72-1,.60(5H,m), 1.49-1.43(2H,q), 1.24-1.10(4H,m), 0.9-0.85(2H,m)

Mass (m/z):400,401,402and 403

### EXAMPLE 36

### 1-(2-Phenylamino-2-oxo ethyl-3-({2-(1-oxo-3-cyclohexyl)-propyl}-hydrazino-carbonyl}-pyridinium bromide.(Compound No:35);

Yield:25%

m.p.:234-236 ° C. (dec)

IR (KBr, cm⁻¹):1689, 1652and 1625

¹HNMR(DMSO d₆, 400 MHz)δ:11.11(1H,s), 10.95(1H,s), 10.23(1H,s), 9.56(1H,s), 9.23-9.21(1H,d), 9.06-9.04(1H,d), 8.38-8.35(1H,m), 7.62-7.60(2H,d), 7.37-7.33(2H,t), 7.12-7.09(1H,t), 5.75(2H,s), 2.25-2.22(2H,t), 1.72-1.60(5H,m) 1.49-1.43(2H,m), 1.25-1.10(4H,m), 0.91-0.83(2H,m)

Mass (m/z):409, 410, 411and 412

### EXAMPLE 37

### 1-(2-Thien-2′-yl-2-oxoethyl)-3-[2-(benzoyloxy)ethylamino carbonyl]-pyridinium bromide (Compound No:36);

Yield:40%

m.p.:125-127° C.

IR (KBr, cm⁻¹):1710and 1675

¹HNMR (DMSO d₆, 400MHz)δ:9.48(1H,s), 9.43-9.41(1H,t), 9.12-9.11 (1H,d), 9.05-9.02(1H,d), 8.40-8.36(1H,m), 8.25-8.20(2H,m), 8.00-7.98(2H,m), 7.68-7.64(1H,m), 7.54-750(2H,m), 7.42-7.40 (1H~m), 6.43(2H,s), 4.46-4.43(2H,t), 3.77-3.73(2H,q)

Mass (m/z):395, 396, 397and 398

### EXAMPLE 38

### 1-(4-Ethoxy-2, 4-dioxobutyl)-3-(2-(benzoyloxy)ethylamino carbonyl)-pyridinium chloride. (Compound No:37);

Yield:35%

m.p.:147-149° C.

IR (KBr, cm⁻¹):1743, 1720, 1680and 1627

¹HNMR (DMSO d₆, 400MHz)δ:9.62-9.59(1H,t), 9.32-9.29(1H,s), 9.05-9.03(1H,d), 8.93-8.90(1H,d), 8.27-8.24(1H,m), 7.92-7.89 (2H,d), 7.59-7.55(1H,m), 7.45-7.41(2H,m), 5.82(2H,s), 4.37-4.34(2H,t), 4.08-4.03(2H,q), 3.80(2H,s), 3.67-3.63(2H,q), 1.15-1.11(3H,t),

Mass (m/z):399, 400and 401

### EXAMPLE 39

### 1-(2′, 4′-Dichloro-phenyl-2-oxoethyl)-3-(2-methoxyethyl aminocarbonyl)-pyridinium bromide. (Compound No:38);

Yield:70%

m.p.:93-95 ° C.

IR (KBr, cm⁻¹):1704, 1664and 1636

¹HNMR (DMSO d₆, 400MHz)δ:9.48(1H,s), 9.29(1H,bs), 9.11-9.08 (2H,m), 8.41-8.38(1H,m), 8.15-8.13(1H,d), 7.92-7.91(1H,t), 7.78-7.75(1H,m), 6.44(2Hs,) 3.52(2H,bs), 3.51(2H,bs), 3.28(3H,s)

Mass (m/z):367,368,369and 370

### EXAMPLE 40

### N,N′-Bis- [3-carbonyl-1-(2-cyclopropylamino-2-oxoethyl) pyridinium]hydrazine dichloride. (Compound No:39):

Yield:40%

m.p. 228-230 ° C.

IR (KBr cm⁻¹):1675, 1636and 1298

¹HNMR (DMSO d₆, 400MHz)δ:11.85(2H,s), 9.59(2H,s), 9.25-9.19 (4H,m), 9.00-8.99(2H,d), 8.39-8.36(2H,m), 5.53(4H,s), 2.73-2.66 (2H,m), 0.78-0.62(4H,m), 0.53-0.49(4H,m)

Mass (m/z) 437, 438and 439

### EXAMPLE 41

### 1-(2-Cyclopropylamino-2-oxoethyl)-3-(2-methoxyethylaminocarbonyl)-pyridinium chloride. (Compound No:40);

Yield:10%

m.p.:122-124 ° C.

IR (KBr, cm⁻¹):1661, 1633, 1549and 121

¹HNMR (DMSOd ₆, 400MHz)δ:9.40(1H,s), 9.08-9.02(2H,m), 8.28-8.25(1H,m), 5.53(2H,s), 3.66-3.61(4H,m), 3.39(3H,s), 2.78-2.74 (1H,m), 0.80-0.75(2H,m), 0.64-0.61(2H,m)

Mass (m/z):278, 279and 280

### EXAMPLE 42

### N-N′-Bis 13-carbonyl-l-2-isopropylamino-2-oxoethyl) pyridinium hydrazine dichloride. (Compound No:41):

Yield:35%

m.p.:114-116 ° C. (dec)

IR (KBr, cm⁻¹):1707, 1668and 1637

¹HNMR (DMSO d(₆, 400MHz)δ:11.84(2H,s), 9.59(2H,s), 9.21-9.18(4H,m), 8.74-8.72(2H,d), 8.39-8.35(2H,m), 5.53(4H,s), 3.92-3.84(2H,m), 1.14-1.02(12H,d)

Mass (m/z):441, 442and 443

### EXAMPLE 43

### 1-(2-Thien-2′yl-2-oxoethyl)-3-(2-(2-chloro-3- pyridoylhydrazinocarbonyl)-pyridinium chloride. (Compound No: 42);

Yield:56%

m.p.:233-235 ° C.

IR (KBr, cm⁻¹):1680, 1637, 1404and 1293

¹HNMR (DMSO d₆, 400 MHz)δ:11.62(1H,s), 11.05(1H,s), 9.62(1H,s), 9.24-9.23(1H,d), 9.18-9.16(1H,d), 8.58-8.56(1H,m), 8.46-8.43(1H,m), 8.26-8.24(2H,m), 8.02-8.00(1H,m), 7.61-7.58(1H,m), 7.43-7.41(1H,m), 6.51(2H,s)

Mass (m/z):401, 402, 403,-404and 405

### EXAMPLE 44

### 1-(2-Isopropylamino-2-oxoethyl)-3-(2-methylsulfonylhydrazinocarbonyl)-pyridinium chloride. (Compound No:43):

Yield:10%

m.p.:227-229 ° C.

IR (KBr, cm⁻¹):1691, 1670, 1566and 1330

¹HNMR (DMSO d(₆, 400MHz)δ:11.55(1H,s), 9.94(1H,s), 9.52(1H,s), 9.16-9.14(1H,m), 9.09-9.07(1H,m), 8.72-8.70(1H,m), 8.34-8.30(1H,m), 5.50(2H,s), 3.89-3.84(1H,m), 3.11(3H,s), 1.13-1.12 (6H,d)

Mass (m/z):315, 316and 317

### EXAMPLE 45

### 1-(2-(1-Pyrrolidinyl)-2-oxoethyl)-3-(methanesulfonyl hydrazino carbonyl) pyridinium chloride (Compound No:44):

Yield:21.00%

m.p.:205-207° C.

IR (KBr, cm⁻¹):1699, 1646and 1589

¹HNMR:(DMSO d₆, 400MHz)δ:11.50(1H,s), 9.94(1H,s), 9.46(1H,s), 9.11-9.06(2H,m), 8.36-8.33(1H,t), 5.75(2H,s), 3.55-3.48(3H1,m), 3.10(3H,s), 2.00-1.95(2H,m), 1.87-1.81(2H,m)

Mass (m/z):327, 328, 329and 330

### EXAMPLE 46

### 1-(2-Thien-2′-yl-2-oxoethyl)-3-(methanesulfony hydrazino carbonyl) pyridinium chloride (Compound No:45);

Yield:31.00%

m.p.:215-217° C.

IR (KBr, cm⁻¹):1685,1666and 1635

¹HNMR:(DMSO d₆, 400MHz)δ:11.49,(1H,s), 9.96(1H,s), 9.55(111s), 9.18(1H,d), 9.10(11,d), 8.43-8.39(1H,t), 8.25-8.22(2H,m), 7.42(1H,t) 6.47(2H,s), 3.09(3H,s).

Mass (m/z):340, 341, 342and 343

### EXAMPLE 47

### N,N′-Bis [3-carbonyl-1-(2-hydroxy-2-oxoethyl) pyridinium]hydrazine dichloride (Compound No:46):

Yield:43.00%

m.p.:235-240° C. (d)

IR (KBr, cm⁻¹):1743, 1700and 1672

¹HNMR (DMSO d₆, 400MHz)δ:11.89(2H,s), 9.69(2H,s), 9.31-9.29 (2H,d), 9.25-9.23(2H,d), 8.43-8.39(2H,t) 5.70(4H,s)

Mass (m/z):360,361,362

### EXAMPLE 48

### 1-(2-Thien-2′-yl-2-oxoethyl)-3-((2-methoxy ethyl) amino carbonyl)-5 -bromo pyridinium chloride (Compound No:47);

Yield:31.00%

m.p.:180-182° C.

IR (KBr, cm⁻¹):1661and 1620

¹HNMR (DMSOd₆, 400MHz)δ:9.58-9.54(2H,d), 9.43-9.39(2H,d), 8.25-8.21(2H,m), 7.41(1H,t), 6.43(2H,s), 3.51(4H,m), 3.29(3H,s).

Mass (m/z):384, 385, 386, 387and 388

### EXAMPLE 49

### 1-(2-Thien-2′-yl-2-oxoethyl)-3-11-oxo-1-(2-methoxycarbonyl) pyridyl]hydrazino Pyridinium chloride (Compound No:48);

Yield:30.00%

m.p.:222-225° C.

IR (KBr, cm⁻¹):1726, 1708 and 1662

¹HNMR (DMSOd₆, 400MHz)δ:11.47(1H,s), 11.23(1H,s), 9.58(1H,s), 9.22-9.15(3H,m), 8.56-8.53(1H,d), 8.46-8.43(1H,t) 8.25-8.21(3H,m), 7.42(1H,t), 6.49(2H,s), 3.95(3H,s)

Mass (m/z):425, 426and 427

### Pharmaceutical Compositions

Pharmaceutical compositions may be prepared with a pharmaceutically effective quantity of compounds of general formula I, individually or in combination. The following pharmaceutical formulations suggested are by way of example alone and in no way restrict the forms in which they can be used.

### Oral formulations

Oral formulations may be administered as solid dosage forms for is example pellets, powders, sachets or discreet units such as tablets or capsules and like. Other orally administered pharmaceutical preparations include monophasic and biphasic liquid dosage forms either in ready to use form or forms suitable for reconstitution such as mixtures, syrups, suspensions or emulsions. The preparations in addition may contain diluents, dispersing agents, buffers, stabilizers, solubilizers, surfactants, preservatives, chelating agents and/or other pharmaceutical additives as are used. Aqueous or non aqueous vehicle or their combination may be used and if desired may contain suitable sweetener, flavoring agent or similar substances. In case of suspension or emulsion a suitable thickening agent or suspending agent or emulsifying agent may be present in addition. Alternatively, the compounds may be administered as such in their pure form unassociated with other additives for example as capsules or sachets. It may also be administered with a vehicle. Pharmaceutical preparations can have a slow, delayed or controlled release of active ingredients as is provided by a matrix or diffusion controlled system.

When the present invention or its salts or suitable complexes is presented as a discreet unit dosage form like tablet, it may contain in addition medically inert excipients as are used in the art. Diluents such as starch, lactose, dicalcium phosphate, talc, magnesium stearate, polymeric substances like methyl cellulose, fatty acids and derivatives, sodium starch glycollate, etc. may also be used.

### EXAMPLE 50

### Preparation of oral dosage form: A typical tablet has the following composition: Active ingredient of formula I as given above Lactose 135 mg Starch 76 mg Polyvinyl pyrolidone (K-30) 2 mg Talc 1.5 mg Magnesium Stearate 1.0 mg

### Parenteral Formulations

For parenteral administration, the compounds or their salts or suitable complexes thereof may be present in a sterile vehicle which may be an aqueous or non aqueous vehicle or a combination thereof The examples of vehicles are water, ethyl oleate, oils and derivatives of polyols, glycols and their derivatives. It may contain additives common in injectable preparations like stabilizers, solubilizers, pH modifiers, buffers, antioxidants, cosolvents, complexing agents, tonicity modifiers, etc.

Some suitable additives are for example tartrate, citrate or similar buffers, alcohol, sodium chloride, dextrose and high molecular weight polymers. Another alternative is sterile powder reconstitution. The compound may be administered in the form of injection for more than once daily administration, or intravenous infusion/drip or suitable depot preparation.

### EXAMPLE 51

### Preparation suitable for parenteral administration has the following composition: Active ingredient of formula I as given above Polyethylene glycol (400 ) 0.75 ml Sodium metabisulphite 0.01% Isotonic saline/ WFI q.s.

### Other Formulations.

For the dermatological application and for the discoloration of teeth, the recommended formulations are lotions, oral rinse and toothpaste containing appropriate amount of the compounds of the general formula I.

The above examples are presented by way of illustration alone and in no way limit the scope of the invention.

What is claimed is:

1. A compound represented by general formula (I), and its pharmaceutically acceptable salts: 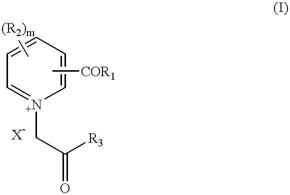
wherein
R₁is -R₄-R₅or -N(R₇) N (R₇) R₉;
R₄is selected from the group consisting of -N(R₇)R₆₀-, -N(R₇)R₆N(R₇),-OR ₆O, and -OR₆N(R₇)-, where R₆is alkyl;
R₅is selected from the group consisting of alkyl, aryl including heteroaryl, -COR₇, -SO₂R₇, -C(S) NHR_{7,}-C(NH)NHR₇, -COR₁₀, 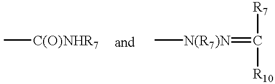
where R₇is selected from the group consisting of H, alkyl and aryl including heteroaryl, provided R₇may be the same or different for R and R₃in the same compound;
R₂is selected from the group consisting of F, Cl, Br, I, OR₇, NO₂, alkyl, aryl including heteroaryl, fornyl, acyl, C(O)NR₇R₁₀, C(O)OR₇,NR₇R₁₀, N=C(R₇)(R₁₀), SR₇, SO₂NH₂, SO₂ alkyl and SO2aryl; m is 0, 1or 2;
R₃is selected from the group consisting of R₇, OR₇, N(R₇) R₁₀), N=C(R₇)(R₁₀), N(R₇) N(R₇)(R₁₀), N(R₇)N=C(R₇)(R₁₀) and CH(R₇)C(O)R₈where R₈is selected from the group consisting of R₇, OR₇ and NR₇R₁₀;
R₉ is selected from the group consisting of hydrogen, alkyl, aryl including heteroaryl, C(O)R₁₀, -SO2R₁₀, C(S)NHR₁₀, C(NH) NH (R₁₀) and C(O) NHR₁₀;
R₁₀ is selected for the group consisting of H, alkyl and aryl, including heteroaryl and in each case may be the same or different from substituent R₇, provided R₁₀ may be the same or different for R₁ and R₃ in the same compound;
X is selected from group consisting of a halide ion, acetate ion, perchlorate ion, sulfonate ion, oxalate ion, citrate ion, tosylate ion, maleate ion, mesylate ion, carbonate ion, sulfite ion, phosphoric hydrogen ion, phosphonate ion, phosphate ion, BF₄⁻and PF₆⁻; with proviso that,
(i) when two alkyl groups are present on the same carbon or nitrogen, they may be linked together to form a cyclic structure, and
(ii) the nitrogen of heteroaryl ring of R₁₀, when present, may be quaternized.

2. The compound as claimed in claim 1, wherein -C(O)R₁ group is at position 3 or 4.

3. The compound as claimed in claim 2, wherein the position for -C(O)R₁ group is at position 3.

4. The compound as claimed in claims 1, wherein m is 0 or 1.

5. The compound as claimed in claim 2, wherein m is 0 or 1.

6. The compound as claimed in claim 3, wherein m is 0 or 1.

7. The compound as claimed in claim 1, wherein m is 0.

8. The compound as claimed in claim 2, wherein m is 0.

9. The compound as claimed in claim 3, wherein m is 0.

10. The compound as claimed in claim 1, wherein X is a halide ion.

11. The compound as claimed in claim 1, which is selected from the group consisting of the following compounds:
(a) N,N′-bis[3-carbonyl-1-(2-thien -2′-yl-2-oxoethyl) -3-pyridinium] hydrazine dibromide and pharmaceutically acceptable salts thereof,
(b) 1-(2-ethoxy -2-oxoethyl) -3-(2-(2-pyridyl)hydrazinocarbonyl) pyridinium bromide and pharmaceutically acceptable salts thereof,
(c) 1-(2-ethoxy -2-oxoethyl) -3-(2-(benzoyloxy) ethylamino carbonyl) pyridinium bromide and pharmaceutically acceptable salts thereof,
(d) N,N′-bis[3-carbonyl-1-(2-phenyl-2-oxoethyl)pyridinium]hydrazine dibromide and pharmaceutically acceptable salts thereof,
(e) 1-(2-phenyl-2-oxoethyl)-3-(hydrazinocarbonyl)pyridinium bromide and pharmaceutically acceptable salts thereof,
(f) 1-(2′-thien -2′-yl -2-oxoethyl) -3-(methanesulfonyl hydrazinocarbonyl) pyridinium bromide and pharmaceutically acceptable salts thereof,
(g)N,N′-bis[3-carbonyl-1-(2-(2′, 4′-dichlorophenyl)-2-oxoethyl) pyridinium] hydrazine dibromide and pharmaceutically acceptable salts thereof,
(h) 1-(2-phenyl -2-oxoethyl) -3-(methanesulfonyl hydrazinocarbonyl) pyridinium bromide and pharmaceutically acceptable salts thereof,
(i) 1-(2-ethoxy -2-oxoethyl) -3-(methanesulfonyl hydrazinocarbonyl) pyridinium bromide and pharmaceutically acceptable salts thereof,
(j) 1-(2-phenyl-2-oxoethyl)-3-(phenylsulfonylhydrazino carbonyl) pyridinium bromide and pharmaceutically acceptable salts thereof,
(k) 1-(2-phenyl-2-oxoethyl)-2-chloro-3-(phenylsulfonyl hydrazino carbonyl) pyridinium bromide and pharmaceutically acceptable salts thereof,
(l) 1-(2-thien -2′-yl -2-oxoethyl)-4-(2-(benzoyloxy) ethyl aminocarbonyl) pyridmium bromide and pharmaceutically acceptable salts thereof,
(m) 1-(2-(2,′,4′-dichlorophenyl) -2-oxoethyl) -3-(2-(benzoyloxy) ethylaminocarbonyl) pyridinium bromide and pharmaceutically acceptable salts thereof,
(n) 1-(2-phenyl -2-oxoethyl) -3-(2-(acetoxy) ethyloxy) carbonyl) pyridinium bromide and pharmaceutically acceptable salts thereof,
(o) 1-(2-ethoxy -2-oxoethyl) -3-(2-(benzoyloxy) ethyloxy carbonyl) pyridinium bromide and pharmaceutically acceptable salts thereof,
(p) 1-(2-phenylamino-2-oxoethyl)4-(phenylsulfonyl hydrazino carbonyl)pyridinium chloride and pharmaceutically acceptable salts thereof,
(q) 1-(2-(2,′,4′-dichlorophenyl)-2-oxoethyl)-3-(2(methoxy) ethyloxycarbonyl) pyridinium bromide and pharmaceutically acceptable salts thereof,
(r) 1-(2-phenylamino-2-oxoethyl)-3-((benzoyloxy) ethylaminocarbonyl) pyridinium chloride and pharmaceutically acceptable salts thereof,
(s) 1-(2-thien-2′-yl-2-oxoethyl)-3-(phenylaminocarbonyl hydrazinocarbonyl) pyridinium bromide and pharmaceutically acceptable salts thereof,
(t) 1-(2-phenyl-2-oxoethyl)-3-(2-(acetoxy) ethylaminocarbonyl) pyridinium bromide and pharmaceutically acceptable salts thereof,
(u) 1-(2-phenylamino-2-oxoethyl)-3-(phenyl sulfonyl hydrazino carbonyl) pyridinium chloride and pharmaceutically acceptable salts thereof,
(v) 1-(2-phenylamino-2-oxoethyl)-3-((4-methylphenyl)sulfonyl hydrazinocarbonyl) pyridinium chloride and pharmaceutically acceptable salts thereof,
(w) 1-(2-phenyl-2-oxoethyl)-3-(2-(benzoyloxy)ethyloxy carbonyl) pyridinium bromide and pharmaceutically acceptable salts thereof,
(x) 1-(2-thien-2′-yl-2-oxoethyl)-3-(phenylcarbonyl hydrazino carbonyl) pyridinium bromide and pharmaceutically acceptable salts thereof,
(y) 1-(2-ethoxy-2-oxoethyl)-3-((phenylmethyl)sulfonyl hydrazino carbonyl)pyridinium bromide and pharmaceutically acceptable salts thereof and
(z) 1-(2-phenyl-2-oxoethyl)-3-((phenylmethyl)sulfonyl hydrazino carbonyl)pyridinium bromide and pharmaceutically acceptable salts thereof.

12. The compound as claimed in claim 1, which is selected from the group consisting of the following compounds:
(aa) N,N′-bis [3-carbonyl-I-(2-furan-2′-yl-2-oxoethyl) pyridinium] hydrazine dibromide and pharmaceutically acceptable salts thereof,
(ab) N,N′-bis [3-carbonyl -1-(2-thien-2′-yl-2-oxoethyl) pyridinium] hydrazine dichloride and pharmaceutically acceptable salts thereof,
(ac) N,N′-bis-[3-carbonyl-1-(2-cyclopropylamino-2-oxoethyl) pyridinium] hydrazine dichloride and pharmaceutically acceptable salts thereof,
(ad) 1-(2′,4′-dichloro-phenyl-2-oxoethyl)-3-(2-methoxyethyl aminocarbonyl)-pyridinium bromide and, pharmaceutically acceptable salts thereof,
(ae) 1-(2-thien-2′-yl-2-oxoethyl)-3-((2-methoxy ethyl) amino carbonyl)-5-bromo pyridinium chloride and pharmaceutically acceptable salts thereof,
(af) 1-(2-thien-2′-yl-2-oxoethyl)-3-(methanesulfonyl hydrazino carbonyl) pyridinium chloride and pharmaceutically acceptable salts thereof,
(ag) 1-(2-thien-2′yl-2-oxoethyl)-3-(2-(2-chloro-3-pyridoylhydrazinocarbonyl)-pyridinium chloride and pharmaceutically acceptable salts thereof,
(ah) 1-(2-cyclopropylamino-2-oxoethyl)-3-(2-methoxyethylaminocarbonyl)-pyridinium chloride and pharmaceutically acceptable salts thereof,
(ai) 1(2-isopropylamino-2-oxoethyl)-3-(2-methylsulfonylhydrazinocarbonyl)-pyridinium chloride and pharmaceutically acceptable salts thereof,
(aj) 1-(2-phenylamino-2-oxo ethyl)-3-({2-(1-oxo-3-cyclohexyl)-propyl}-hydrazino-carbonyl)-pyridinium bromide and pharmaceutically acceptable salts thereof,
(ak) 1-(2-thien-2′-yl-2-oxoethyl)-3-[2-(benzoyloxy)ethylamino carbonyl]-pyridinium bromide and pharmaceutically acceptable salts thereof,
(al) 1-(4-ethoxy-2, 4-dioxobutyl)-3-(2-(benzoyloxy)ethylamino carbonyl)-pyridinium chloride and pharmaceutically acceptable salts thereof, and
(am) 1-(2-thien-2′-yl-2-oxoethyl)-3-[l-oxo-1-(2-methoxy carbonyl) pyridyl] hydrazino pyridinium chloride and pharmaceutically acceptable salts thereof.

13. A process for the preparation of the compound represented by general formula (I) as claimed in claim 1, which comprises preparing a substituted pyridine, followed by quaternizing the substituted pyridine, with a quaternizing reagent in an alcoholic and/or high boiling solvent under reflux for 6-48hrs. to give the desired compound.

14. The compound of general formula I as defined in claim 1, for use as a medicament in the treatment of diabetic complications and aging-related diseases.

15. The compound of general formula (I), as defined in claim 1, for use as a medicament in the treatment of kidney disease, nerve damage, retinopathy, atherosclerosis, microangiopathy, endothelial dysfunctions, dermatological conditions and discoloration of teeth.

16. A pharmaceutical composition comprising a pharmaceutically effective amount of one or more compounds represented by general formula (I), ad defined in claim 1 or pharmaceutically acceptable salt(s) thereof in admixture with a pharmaceutically acceptable carrier, diluent, solvent or excepient.

17. The pharmaceutical composition as claimed in claim 16, in the form of an oral formulation.

18. The pharmaceutical composition as claimed in claim 16, wherein said acceptable carier, diluent, solvent or excepient is selected from group consisting of starch, lactose, polyvinyl pyrolidone (K-30), talc and magnesium stearate.

19. The pharmaceutical composition as claimed in claim 16 in the form of a parenteral formulation.

20. A method for the preparation of a parenteral formulation as claimed in claim 19, which comprises dissolving one or more compounds represented by general formula (I), as defined in claim 1, in polyethylene glycol 400 and diluting the solution so obtained, with an isotonic solution or water to a desired concentration.

21. Pharmaceutical composition as claimed in claim 16, in the form of a lotion, oral rinse and toothpaste.

22. A method for treating a diabetic patient by breaking a preformed AGE, within said patient, which comprises, administering an effective amount of a compound represented by general formula (I) as claimed in claim 1, either singly, or in combination with other drugs for antidiabetic therapy.

23. A method of preventing or treating diseases caused by diabetes and aging related complications, which comprises, administering to a patient in need thereof, an effective amount of a compound represented by general formula (I), as claimed in claim 1, either singly or in combination with a pharmaceutically acceptable carrier, diluent or excepient.

24. The method as claimed in claim 23, wherein the disease prevented or treated is a nephrological disorder, neurological disorder, atherosclerosis, retinal disorder, dermatological disorder, non-enzymatic browning of oral cavity, endothelial or other organ dysfunction and growth impairment.

25. The method as claimed in claim 22, wherein said compound is selected from the group consisting of:
(a) N,N′-bis[3-carbonyl-1-(2-thien -2′-yl -2-oxoethyl) -3-pyridinium] hydrazine dibromide and pharmaceutically acceptable salts thereof,
(b) 1-(2-ethoxy -2-oxoethyl) -3-(2-(2-pyridyl)hydrazinocarbonyl) pyridinium bromide and pharmaceutically acceptable salts thereof,
(c) 1-(2-ethoxy -2-oxoethyl) -3-(2-(benzoyloxy)ethylamino carbonyl) pyridinium bromide and pharmaceutically acceptable salts thereof,
(d) N,N′-bis[3-carbonyl-1-(2-phenyl-2-oxoethyl)pyridinium]hydrazine dibromide and pharmaceutically acceptable salts thereof,
(e) 1-(2-phenyl-2-oxoethyl)-3-(hydrazinocarbonyl)pyridinium bromide and pharmaceutically acceptable salts thereof,
(f) 1-(2-thien -2′-yl -2-oxoethyl) -3-(methanesulfonyl hydrazinocarbonyl) pyridinium bromide and pharmaceutically acceptable salts thereof,
(g) N,N′-bis [3-carbonyl -1-(2-(2′,4′-dichlorophenyl) -2-oxoethyl) pyridinium] hydrazine dibromide and pharmaceutically acceptable salts thereof,
(h) 1-(2-phenyl -2-oxoethyl) -3-(methanesulfonyl hydrazinocarbonyl) pyridinium bromide and pharmaceutically acceptable salts thereof,
(i) 1-(2-ethoxy -2-oxoethyl) -3-(methanesulfonyl hydrazinocarbonyl) pyridinium bromide and pharmaceutically acceptable salts thereof,
(j) 1-(2-phenyl-2-oxoethyl)-3-(phenylsulfonylhydrazinocarbonyl) pyridinium bromide and pharmaceutically acceptable salts thereof,
(k) 1-(2-phenyl-2-oxoethyl)-2-chloro-3-(phenylsulfonylhydrazino carbonyl) pyridinium bromide and pharmaceutically acceptable salts thereof,
(l) 1-(2-thien -2′-yl -2-oxoethyl)-4-(2-(benzoyloxy) ethylaminocarbonyl) pyridinium bromide and pharmaceutically acceptable salts thereof,
(m) 1-(2-(2,4-dichlorophenyl)-2-oxoethyl)-3-(2-(benzoyloxy)ethylamino carbonyl) pyridinium bromide and pharmaceutically acceptable salts thereof,
(n) 1-(2-phenyl -2-oxoethyl) -3-(2-(acetoxy) ethyloxy carbonyl) pyridinium bromide and pharmaceutically acceptable salts thereof,
(o) l-(2-ethoxy -2-oxoethyl) -3-(2-(benzoyloxy) ethyloxy carbonyl) pyridinium bromide and pharmaceutically acceptable salts thereof,
(p) 1-(2-phenylamino-2-oxoethyl)-4-(phenylsulfonyl hydrazino carbonyl) pyridinium chloride and pharmaceutically acceptable salts thereof,
(q) 1-(2-(2′,4′-dichlorophenyl)-2-oxoethyl)-3-(2(methoxy) ethyloxycarbonyl) pyridinium bromide and pharmaceutically acceptable salts thereof,
(r) 1-(2-phenylamino-2-oxoethyl)-3-((benzoyloxy) ethylaminocarbonyl) pyridinium chloride and pharmaceutically acceptable salts thereof,
(s) 1-(2-thien-2′-yl-2-oxoethyl)-3-(phenylaminocarbonyl hydrazinocarbonyl) pyridinium bromide and pharmaceutically acceptable salts thereof,
(t) 1-(2-phenyl-2-oxoethyl)-3-(2-(acetoxy) ethylaminocarbonyl) pyridinium bromide and pharmaceutically acceptable salts thereof,
(u) 1-(2-phenylamino-2-oxoethyl)-3-(phenyl sulfonyl hydrazino carbonyl) pyridinium chloride and pharmaceutically acceptable salts thereof,
(v) 1-(2-phenylamino-2-oxoethyl)-3-((4-methylphenyl)sulfonyl hydrazino carbonyl) pyridinium chloride and pharmaceutically acceptable salts thereof,
(w) 1-(2-phenyl-2-oxoethyl)-3-(2-(benzoyloxy)ethyloxy carbonyl) pyridinium bromide and pharmaceutically acceptable salts thereof,
(x) 1-(2-thien-2′-yl-2-oxoethyl)-3-(phenylcarbonyl hydrazino carbonyl) pyridmium bromide and pharmaceutically acceptable salts thereof,
(y) 1-(2-ethoxy-2-oxoethyl)-3-((phenylmethyl)sulfonyl hydrazino carbonyl) pyridinium bromide and pharmaceutically acceptable salts thereof and
(z) 1-(2-phenyl-2-oxoethyl)-3-((phenylmethyl) sulfonyl hydrazinocarbonyl) pyridinium bromide and pharmaceutically acceptable salts thereof,

26. The method as claimed in claim 22, wherein said compound is selected from the group consisting of:
(aa) N,N′-bis [3-carbonyl-1-(2-furan-2′-yl-2-oxoethyl) pyridinium] hydrazine dibromide and pharmaceutically acceptable salts thereof,
(ab) N,N′-bis [3-carbonyl -1-(2-thien-2′-yl-2-oxoethyl) pyridinium] hydrazine dichloride and pharmaceutically acceptable salts thereof,
(ac) 1-(2-phenylamino-2-oxo ethyl)-3-({2-(1-oxo-3-cyclohexyl)- propyl} -hydrazino-carbonyl)-pyridinium bromide and pharmaceutically acceptable salts thereof, thereof,
(ae) 1-(4-ethoxy-2, 4-dioxobutyl)-3-(2-(benzoyloxy)ethylamino carbonyl)-pyridinium chloride and pharmaceutically acceptable salts thereof,
(af) 1-2′,4′ -dichloro-phenyl-2-oxoethyl)-3-(2-methoxyethyl aminocarbonyl)-pyridinium bromide and pharmaceutically acceptable salts thereof,
(ag) N,N′-bis-[3-carbonyl-1-(2-cyclopropylamino-2-oxoethyl) pyridinium] hydrazine dichloride and pharmaceutically acceptable salts thereof,
(ah) 1-(2-cyclopropylamino-2-oxoethyl)-3-(2-methoxyethylaminocarbonyl)-pyridinium chloride and pharmaceutically acceptable salts thereof,
(ai) 1-(2-thien-2′yl-2-oxoethyl)-3-(2-(2-chloro-3-pyridoylhydrazinocarbonyl)-pyridinium chloride and pharmaceutically acceptable salts thereof,
(aj) 1-(2-isopropylamino-2-oxoethyl)-3-(2-methylsulfonylhydrazinocarbonyl)-pyridinium chloride and pharmaceutically acceptable salts thereof,
(ak) 1-(2-thien-2′-yl-2-oxoethyl)-3-(methanesulfonylhydrazino carbonyl) pyridinium chloride and pharmaceutically acceptable salts thereof,
(al) 1-(2-thien-2′-yl-2-oxoethyl)-3-((2-methoxy ethyl) amino carbonyl)-5 -bromo pyridinium chloride and pharmaceutically acceptable salts thereof, and
(am) 1-(2-thien-2′-yl-2-oxoethyl)-3-[ 1-oxo-1-(2-methoxycarbonyl) pyridyl] hydrazino pyridinium chloride and pharmaceutically acceptable salts thereof.

27. The method as claimed in claim 23, wherein said compound is selected from the group consisting of:
(a) N,N′-bis[3-carbonyl-1-(2-thien -2′-yl -2-oxoethyl) -3-pyridinium ]hydrazine dibromide and pharmaceutically acceptable salts thereof,
(b) 1-(2-ethoxy -2-oxoethyl) -3-(2-(2-pyridyl)hydrazinocarbonyl) pyridimium bromide and pharmaceutically acceptable salts thereof,
(c) 1-(2-ethoxy -2-oxoethyl) -3-(2-(benzoyloxy)ethylamino carbonyl) pyrdinium bromide and pharmaceutically acceptable salts thereof,
(d) N,N′-bis[3-carbonyl-1-(2-phenyl-2-oxoethyl)pyridinium]hydrazine dibromide and pharmaceutically acceptable salts thereof,
(e) 1-(2-phenyl-2-oxoethyl)-3-(hydrazinocarbonyl)pyridinium bromide and pharmaceutically acceptable salts thereof,
(f) 1-(2-thien -2′-yl -2-oxoethyl) -3-(methanesulfonyl hydrazinocarbonyl) pyridinium bromide and pharmaceutically acceptable salts thereof,
(g) N,N′-bis [3-carbonyl -1-(2-(2 ,41-dichlorophenyl) -2-oxoethyl) pyridinium] hydrazine dibromide and pharmaceutically acceptable salts thereof,
(h) 1-(2-phenyl -2-oxoethyl) -3-(methanesulfonyl hydrazinocarbonyl) pyridinium bromide and pharmaceutically acceptable salts thereof,
(i) 1-(2-ethoxy -2-oxoethyl) -3-(methanesulfonyl hydrazinocarbonyl) pyridinium bromide and pharmaceutically acceptable salts thereof,
(j) 1-(2-phenyl-2-oxoethyl)-3-phenylsulfonylhydrazinocarbonyl) pyridinium bromide and pharmaceutically acceptable salts thereof,
(k) -(2-phenyl-2-oxoethyl)-2-chloro-3-(phenylsulfonylhydrazino carbonyl) pyridinium bromide and pharmaceutically acceptable salts thereof,
(l) 1-(2-thien -2′-yl -2-oxoethyl)-4-(2-(benzoyloxy) ethylaminocarbonyl) pyridimium bromide and pharmaceutically acceptable salts thereof,
(m) 1-(2-(2,4-dichlorophenyl)-2-oxoethyl)-3-(2-(benzoyloxy)ethylamino carbonyl) pyrdinium bromide and pharmaceutically acceptable salts thereof,
(n) 1-(2-phenyl -2-oxoethyl) -3-(2-(acetoxy) ethyloxy carbonyl) pyridmium bromide and pharmaceutically acceptable salts thereof,
(o) 1-(2-ethoxy -2-oxoethyl) -3-(2-(benzoyloxy) ethyloxy carbonyl) pyridinium bromide and pharmaceutically acceptable salts thereof,
(p) 1-(2-phenylamino-2-oxoethyl)-4-(phenylsulfonyl hydrazino carbonyl) pyridmium chloride and pharmaceutically acceptable salts thereof,
(q) 1-(2-(2′,4′-dichlorophenyl)-2-oxoethyl)-3-(2(methoxy) ethyloxycarbonyl) pyridmium bromide and pharmaceutically acceptable salts thereof,
(r) 1-(2-phenylamino-2-oxoethyl)-3-((benzoyloxy) ethylaminocarbonyl) pyridinium chloride and pharmaceutically acceptable salts thereof,
(s) 1-(2-thien-2′-yl-2-oxoethyl)-3-(phenylaminocarbonyl hydrazinocarbonyl) pyridinium bromide and pharmaceutically acceptable salts thereof,
(t) 1-(2-phenyl-2-oxoethyl)-3-(2-(acetoxy) ethylaminocarbonyl) pyridinium bromide and pharmaceutically acceptable salts thereof,
(u) 1-(2-phenylamino-2-oxoethyl)-3-(phenyl sulfonyl hydrazino carbonyl) pyridinium chloride and pharmaceutically acceptable salts thereof,
(v) 1-(2-phenylamino-2-oxoethyl)-3-((4-methylphenyl)sulfonyl hydrazino carbonyl) pyridmium chloride and pharmaceutically acceptable salts thereof,
(w) 1-(2-phenyl-2-oxoethyl)-3-(2-(benzoyloxy)ethyloxy carbonyl) pyridiwum bromide and pharmaceutically acceptable salts thereof,
(x) 1-(2-thien-2′-yl-2-oxoethyl)-3-(phenylcarbonyl hydrazino carbonyl) pyridinium bromide and pharmaceutically acceptable salts thereof,
(y) 1-(2-ethoxy-2-oxoethyl)-3-((phenylmethyl)sulfonyl hydrazino carbonyl) pyridinium bromide and pharmaceutically acceptable salts thereof and
(z) 1-(2-phenyl-2-oxoethyl)-3-((phenylmethyl) sulfonyl hydrazinocarbonyl) pyridinium bromide and pharmaceutically acceptable salts thereof,

28. The method as claimed in claim 23, wherein said compound is selected from the group consisting of:
(aa) N,N′-bis [3-carbonyl-1-(2-furan-2′-yl-2-oxoethyl) pyridinium] hydrazine dibromide and pharmaceutically acceptable salts thereof,
(ab) N,N′-bis [3-carbonyl -1-(2-thien-2′-yl-2-oxoethyl) pyridinium] hydrazine dichloride and pharmaceutically acceptable salts thereof,
(ac) 1-(2-phenylamino-2-oxo ethyl)-3-({2-(l-oxo-3-cyclohexyl)-propyl } -hydrazino-carbonyl)-pyridmium bromide and pharmaceutically acceptable salts thereof,
(ad) 1-(2-thien-2′-yl-2-oxoethyl)-3-[2-(benzoyloxy)ethylamino carbonyl]-pyridinium bromide and pharmaceutically acceptable salts thereof,
(ae) 1-(4-ethoxy-2, 4-dioxobutyl)-3-(2-(benzoyloxy)ethylamino carbonyl)-pyridinium chloride and pharmaceutically acceptable salts thereof,
(af) 1-(2′,4′-dichloro-phenyl-2-oxoethyl)-3-(2-methoxyethyl aminocarbonyl)-pyridinium bromide and pharmaceutically acceptable salts thereof,
(ag) N,N′-bis-[3-carbonyl-1-(2-cyclopropylamino-2-oxoethyl) pyridinium]hydrazine dichloride and pharmaceutically acceptable salts thereof,
(ah) 1-(2-cyclopropylamino-2-oxoethyl)-3-(2-methoxyethylaminocarbonyl)-pyrdinium chloride and pharmaceutically acceptable salts thereof,
(ai) 1-(2-thien-2′yl-2-oxoethyl)-3-(2-(2-chloro-3-pyridoylhydrazinocarbonyl)-pyridinium chloride and pharmaceutically acceptable salts thereof,
(aj) 1-(2-isopropylamino-2-oxoethyl)-3-(2-methylsulfonylhydraziocarbonyl)-pyridinium chloride and pharmaceutically acceptable salts thereof,
(ak) 1-(2-thien-2′-yl-2-oxoethyl)-3-(methanesulfonylhydrazino carbonyl) pyridinium chloride and pharmaceutically acceptable salts thereof,
(al) 1-(2-thien-2′-yl-2-oxoethyl)-3-((2-methoxy ethyl) amino carbonyl)-5 -bromo pyridinium chloride and pharmaceutically acceptable salts thereof, and
(am) 1-(2-thien-2′-yl-2-oxoethyl)-3-[1-oxo-1-(2-methoxycarbonyl) pyridyl] hydrazino pyridinium chloride and pharmaceutically acceptable salts thereof.
